(19) United States
(12) Patent Application Publication
(10) Pub. No.: US 2021/0145748 A1
(43) Pub. Date: May 20, 2021

(54) PROCESSING SYSTEM FOR POWDERS, AND METHOD FOR DECONTAMINATION OF SUCH A PROCESSING SYSTEM

(71) Applicant: Harro Hoefliger Verpackungsmaschinen GmbH, Allmersbach im Tal (DE)

(72) Inventors: Oliver Rau, Allmersbach im Tal (DE); Bernhard Brugger, Allmersbach im Tal (DE); Michael Jeschke, Allmersbach im Tal (DE); Jonas Hoepfer, Allmersbach im Tal (DE); Reiner Wurst, Allmersbach im Tal (DE)

(21) Appl. No.: 16/951,791

(22) Filed: Nov. 18, 2020

(30) Foreign Application Priority Data
Nov. 19, 2019 (EP) 19 210 187.1

(51) Int. Cl. A61K 9/14; B01L 1/04; B08B 9/032

(52) U.S. Cl. CPC A61K 9/14; B01L 2300/10; B08B 9/0321; B01L 1/04

(57) ABSTRACT

The disclosure relates to a processing system for in particular pharmaceutical powders, and to a method for decontamination of such a processing system. The processing system includes a processing device for the powder, and a closed housing with an inner production space in which the processing device is arranged. The processing system further includes a steam generator via which water vapor is introduced into the production space in such a quantity that air supersaturated with water vapor develops there at least in some parts. Any powder residues present are bound via water since, on account of the supersaturation, water condenses in the production space. The condensed water, including powder residues bound therein, is removed, in particular with the housing opened.

### CROSS REFERENCE TO RELATED APPLICATION

This application claims priority of European patent application no. 19 210 187.1, filed Nov. 19, 2019, the entire content of which is incorporated herein by reference.

### FIELD OF THE INVENTION

The disclosure relates to a processing system for in particular pharmaceutical powders and to a method for decontamination of such a processing system.

### BACKGROUND OF THE INVENTION

In the pharmaceutical industry, some highly effective powders are processed, for example by being filled into hard gelatin capsules or pressed into tablet form. At a given concentration, such highly effective powders may have a toxic action. In the processing of such powders, it therefore has to be ensured that the machine operator and the environment are not overly exposed or even endangered.

According to the prior art, such powders are processed in isolated rooms. A corresponding processing device is arranged in a production space tightly enclosed by a housing. Such housings are also referred to as containments. While containment processing systems provide reliable protection of the environment during normal operation, a further challenge is also to be able to open the closed housing as and when necessary. A problem lies in the contamination of the inner production space, and of the machine parts arranged therein, with unavoidable powder residues.

For example, when production is interrupted or after a production cycle has been completed, it is necessary for intensive cleaning to be carried out to remove the powder residues before the housing is opened.

This can be done, according to the prior art, by intensive rinsing with water, if appropriate with addition of cleaning agents. The rinse water running off is tested for powder constituents. Only when a sufficiently low concentration is found can the housing door be opened to permit access to the production space. Such a process is referred to as washing in place (WIP).

The WIP method requires specially configured machines with suitably closed housings. Machine and housing must be absolutely leaktight, particularly with respect to the rinse water used, so that no contaminated rinse water can escape. With the large number of movable parts involved, this is difficult to achieve, since it is not just the housing and its door that have to be reliably sealed off but also the movable parts of the processing device such as shaft bearings, actuation ducts or the like. The use of standard machines is thus excluded. On the other hand, the procurement and operation of WIP-capable machines entail high investment costs and operating costs and also make production inflexible. There are also high costs involved in disposal of the rinse water.

Binding of the powder by a spray mist is in some cases accepted as a cost-effective alternative to the WIP method. This involves the use of a two-substance nozzle in which water is atomized via compressed air in order to wet or moisten the contaminated surfaces. Powder residues adhering to the surface are bound by the moisture. This permits opening of the containment and subsequent manual cleaning. However, it has been found that the geometrically complex structure of the machine prevents all regions from being reached by the spray mist and prevents the power residues located there from being bound.

### SUMMARY OF THE INVENTION

It is an object of the invention to provide a processing system such way that, along with low investment costs, simple and safe cleaning is possible.

This object can, for example, be achieved by a processing system for pharmaceutical powders having the features of a processing device for the powder; a closed housing defining an inner production space; the processing device being arranged in the inner production space enclosed by the housing; and, a steam generator configured to introduce water vapor into the inner production space in such a way that air supersaturated with water vapor develops in at least some parts of the inner production space.

It is a further object of the invention to provide a suitable method for decontamination of such a processing system.

This object can, for example, be achieved by a method for decontamination of a processing system having a processing device for powder; a closed housing defining an inner production space; the processing device being arranged in the inner production space enclosed by the housing; and, a steam generator configured to introduce water vapor into the inner production space. The method includes: introducing water vapor into the inner production space in such a quantity that air supersaturated with the water vapor develops in at least some parts of the inner production space, wherein any powder residues present are bound via water because, on account of the supersaturation, water condenses in the production space; and, removing the condensed water, including powder residues bound therein.

According to the disclosure, a steam generator is provided as part of the processing system. This steam generator and the associated method according to the disclosure are set up such that water vapor can be introduced into the production space in such a way that air supersaturated with water vapor develops there at least in some parts. As a result of the supersaturation, water condenses in the production space, specifically on surfaces of the processing device, and also in the air as a mist. Any powder residues present on the surfaces and/or any powder grains suspended freely in the air are bound by the quantities of condensed water. The condensed water, including the powder residues bound therein, is then removed, and this can be done in particular with the housing opened.

The formation of condensation water is usually undesirable in technology, and this applies in particular in the technical field of powder processing. Going against this ground rule, formation of condensation water is now brought about deliberately according to the disclosure. As preparation for the subsequent cleaning, it exploits the fact that a controlled or specifically directed application of moisture does not have to be carried out. Instead, the formation of condensation water takes place without controlled intervention in every corner of the machine, however inaccessible. Powder residues, which may accumulate in particular in such inaccessible locations, are wetted with water in a reliable and seamless way and are thus bound. Following the condensate formation, the housing or the containment of the machine can thus be opened without danger. Manual cleaning can then take place. The investment cost for supply of steam is low. At the same time, in contrast to WIP configurations, no special sealing measures are needed against escape of wash water. Cleaning of the machine is reliable and safe and can be done with minimal effort.

In an embodiment, a stationary steam nozzle, supplied from the steam generator, is arranged in the production space. In this way, water vapor can be introduced as and when necessary, until a supersaturated air/steam mixture develops. By way of this stationary steam nozzle, the state of supersaturation can be maintained until sufficient condensate formation has been obtained at the relevant locations.

In a further embodiment, the processing system includes a pressure nozzle, which is to be guided manually. The latter can be an air pressure or water pressure nozzle or else a two-substance nozzle via which preliminary cleaning is effected before the actual condensate formation, by means of the reachable locations being blown clean by pressure. In an embodiment, the pressure nozzle is supplied from the steam generator. The water vapor thus introduced in this case has a dual use. Firstly, it serves for the preliminary cleaning by virtue of its pressure action. Secondly, the supplied steam contributes to achieving the supersaturated state and thus the condensate formation. The pressure nozzle, under application of steam, can thus be used as a supplement to the stationary steam nozzle, but also as a replacement for the latter.

In addition, it may be expedient to provide an ultrasonic humidifier via which fine water droplets are introduced into the closed production space, and as a result of which the binding of powder residues, which is brought about by condensed water, is supported.

### BRIEF DESCRIPTION OF THE DRAWING

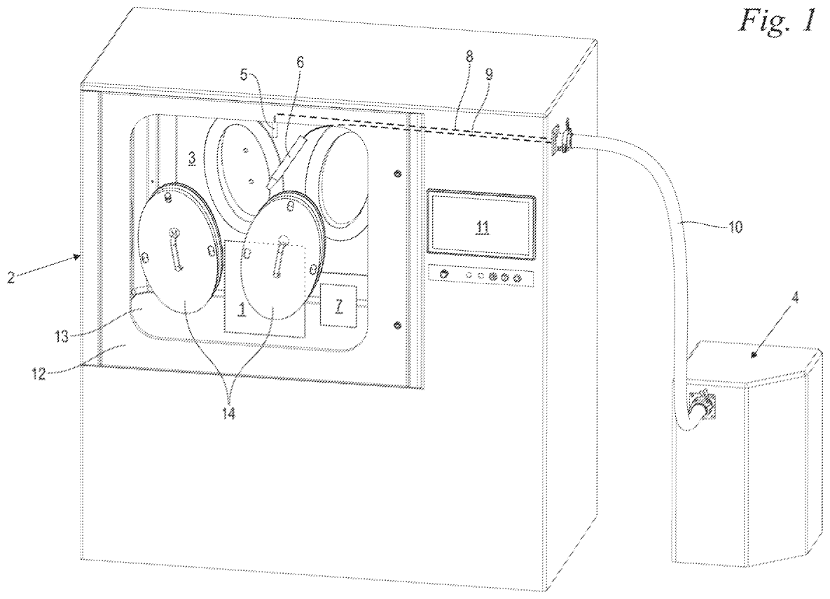

The invention will now be described with reference to the single figure of the drawing (FIG. 1) which shows a perspective view of a processing system configured according to the disclosure for pharmaceutical powders.

### DESCRIPTION OF THE PREFERRED EMBODIMENTS OF THE INVENTION

FIG. 1 shows a perspective view of a processing system configured according to the disclosure for pharmaceutical powders. The processing system includes a processing device 1 (indicated only schematically as a block) for the powder, which processing device 1 is in this case a capsule-filling device, although it can also be a tablet press or the like. The processing system 1 moreover includes a closed housing 2, which encloses an inner production space 3 and seals the latter off hermetically from the outside environment during operation. The processing device 1 is positioned in this inner production space 3 such that no partial quantities of the powder that is to be processed can escape to the outside.

The processing device 1 can be operated from the outside of the housing 2 via a user interface 11. The operation can be visually monitored through a window 13, which is additionally equipped with glove ports 14. When necessary, manual access can be obtained via the glove ports 14, without the housing 2 having to be opened. In the decontaminated state, however, the housing 2 can be opened, for which purpose a door 12 is provided.

During the operation of the processing device 1, powder residues can settle in the inner production space 3, on the inner faces of the housing 2 and also on all surfaces of the processing device 1. In addition, airborne powder particles are suspended in the air of the production space 3. All of these powder residues or powder particles have to be removed by cleaning if necessary. For this purpose, the processing system includes a steam generator 4 in addition to the housing 2 and to the processing device 1 arranged in the inner production space 3. The steam generator 4 is shown as an autonomous unit separate from the housing 2, but it can also be integrated in the housing 2 or in the processing device 1. The steam generator 4 is controlled via the user interface 11. The steam generator 4 is connected to the housing 2 via a medium-conveying hose 10. During operation, that is, for the purpose of decontamination after the end of production or in a production break, hot water vapor, that is, water in its gaseous phase, is generated via the steam generator 4 and is introduced through the medium-conveying hose 10 into the production space 3. As regards its capacity and control, the steam generator 4 is configured such that air supersaturated with water vapor develops in the production space. Thus, in the corresponding method step, water vapor is introduced into the production space 3 in such a quantity that the air present therein anyway is supersaturated with water vapor and consequently leads to condensation of water. The condensation of the water takes place on all the free surfaces within the production space 3, that is, as droplets of mist on powder particles suspended freely in the air contained in the production space, on the inner faces of the housing 2, and on all the free surfaces of the processing device 1. Water vapor continues to be introduced until all the suspended particles of mist droplets are caught and until all surfaces are wetted with condensation water in the liquid phase to such an extent that powder residues adhering to them are bound by the water. The mist droplets, with the powder particles bound therein, form a sediment after a while, that is, settle on the inner faces of the housing 2 and on the free surfaces of the processing device 1.

After condensation and sedimentation have taken place, that is, after binding of the powder residues, the door 12 can be opened and the production space 3, including the processing device 1, can be cleaned. For this purpose, the condensate, with the powder residues bound therein, can be suctioned off and/or wiped off.

In the illustrative embodiment shown, the water vapor is introduced into the production space 3 via a stationary steam nozzle supplied via a steam line 8. The water vapor thus supplied mixes with the air until the desired saturation is achieved at least in some parts, that is, in at least all relevant volume regions. To support the condensation process then initiated and the associated wetting of the surfaces with moisture, an ultrasonic humidifier 7 can optionally be used, which is indicated here only as a schematic block and is positioned in the production space 3. However, the ultrasonic humidifier 7 can also be arranged at another suitable location, for example in the housing of the steam generator 4. In any case, fine water droplets can be generated via the ultrasonic humidifier 7, as and when necessary, and can be discharged into the air contained in the production space 3. These water droplets also bind suspended particles, and they too settle on the free surfaces and contribute to the binding of powder residues.

Prior to the described process of powder binding via condensed water, a preliminary cleaning of the surfaces can optionally take place, with the housing 2 closed. For this purpose, a pressure nozzle 6 is optionally provided, which is supplied via a flexible hose and can be guided manually by way of the glove ports 14. The machine operator can use it to blow the soiled surfaces clean, whereupon the quantities of powder that swirl up are then suctioned off. The pressure nozzle 6 can be, for example, a compressed air nozzle or a multi-substance nozzle for supplying an air/water mixture. In the illustrative embodiment shown, the nozzle is a superheated steam nozzle, which is supplied via a steam line 9 from the steam generator 4.

The superheated steam, impinging with pressure on the corresponding surfaces, leads in the first instance to a preliminary cleaning in the manner described above. In addition, it contributes to increasing the relative air humidity in the production space 3. Accordingly, only quite a small quantity of water vapor then has to be supplied via the stationary steam nozzle 5 until the state of supersaturation and condensation arises. Alternatively, it may also suffice to do without the stationary steam nozzle 5 entirely and to supply the water vapor, both for the preliminary cleaning and for generating the supersaturated condensation state, via the pressure nozzle 6 alone.

It is understood that the foregoing description is that of the preferred embodiments of the invention and that various changes and modifications may be made thereto without departing from the spirit and scope of the invention as defined in the appended claims.

What is claimed is:

1. A processing system for pharmaceutical powders, the processing system comprising:
a processing device for the powder;
a closed housing defining an inner production space;
said processing device being arranged in said inner production space enclosed by said housing; and,
a steam generator configured to introduce water vapor into said inner production space in such a way that air supersaturated with water vapor develops in at least some parts of said inner production space.

2. The processing system of claim 1 further comprising a stationary steam nozzle arranged in said inner production space, wherein said stationary steam nozzle is configured to be supplied by said steam generator.

3. The processing system of claim 1 further comprising a pressure nozzle which is to be guided manually.

4. The processing system of claim 3, wherein said pressure nozzle is configured to be supplied from the steam generator.

5. The processing system of claim 1 further comprising an ultrasonic humidifier for introducing moisture into said inner production space.

6. A method for decontamination of a processing system having a processing device for powder; a closed housing defining an inner production space; the processing device being arranged in the inner production space enclosed by the housing; and, a steam generator configured to introduce water vapor into the inner production space; the method comprising:
introducing water vapor into the inner production space in such a quantity that air supersaturated with the water vapor develops in at least some parts of the inner production space, wherein any powder residues present are bound via water because, on account of the supersaturation, water condenses in the production space; and,
removing the condensed water, including powder residues bound therein.

7. The method of claim 6, wherein the condensed water, including powder residues bound therein, is removed, with the housing opened.

8. The method of claim 6, further comprising preliminary cleaning via a pressure nozzle with the housing closed.

9. The method of claim 8, wherein the pressure nozzle is supplied from the steam generator.

10. The method of claim 6, wherein the binding of powder residues, which is brought about by condensed water, is supported via an ultrasonic humidifier.
